(19) United States
(12) Patent Application Publication
(10) Pub. No.: US 2013/0299661 A1
(43) Pub. Date: Nov. 14, 2013

(54) MEDICAL DEVICE FOR EXTRACORPORAL TREATMENT OF BLOOD WITH FILTER HOLDER

(71) Applicant: B. Braun Avitum AG, Melsungen (DE)

(72) Inventor: ANDREAS SCHADE, Rotenburg (DE)

(21) Appl. No.: 13/888,834

(22) Filed: May 7, 2013

(30) Foreign Application Priority Data
May 11, 2012 (DE) 10 2012 104 157.1

(51) Int. Cl. F16M 13/00; A61M 1/34

(52) U.S. Cl. CPC F16M 13/00; A61M 1/34
USPC 248/313

(57) ABSTRACT

A filter holder for the support of a filter on the casing of a medical device for extra-corporal blood treatment is disclosed. The filter holder has a first grip leg and a second grip leg, which are arranged with respect to each other so that a reception area is formed between the two grip legs. The filter can be inserted into this reception area between the two grip legs, so that the filter is held in the filter holder against the force of gravity. At least one trough- or U-shaped indentation is provided on at least one grip leg, whose cross-section enables the wedging in of a hose. A medical device for extra-corporal blood treatment with such a filter holder for the reception of a filter is also disclosed.

### CROSS REFERENCE TO RELATED APPLICATIONS

This application claims priority to European Application No. DE 10 2012 104 157.1 filed May 11, 2012, the contents of such application being incorporated by reference herein.

### FIELD OF THE INVENTION

The invention concerns a filter holder for the support of a filter on the casing of a medical device for extra-corporal blood treatment, where the filter holder indicates at least a first grip leg and a second grip leg, which are arranged with respect to each other so that a reception area is formed between the two grip legs, into which the filter is capable of being inserted.

The invention further concerns a medical device for extra-corporal blood treatment with such a filter holder.

### BACKGROUND OF THE INVENTION

In the case of medical devices with extra-corporal blood circulation for blood cleaning, blood hose systems are usually attached to the medical device. Hose brackets are helpful here for orientation and sorting. Among other things, hose brackets are also required in the area of the dialyzer holder, which in particular should prevent any kinking of the hoses to/from the dialyzer.

It is known in this case to realize the hose holder function through detachable attachment parts, for example on the casing of the medical device. Detachable attachment parts can also be lost, however, which is disadvantageous. Against this, fixed-assembled attachment parts with hose brackets can break off, which can also be disadvantageous. Furthermore, the arrangement of the hose brackets can be unsatisfactory if hose brackets are required in a certain area such as the dialyzer, however, without positions suitable for that being provided.

### DESCRIPTION OF THE RELATED ART

Furthermore, from DE 24 12 285 A1, for example, a rotating-change support holder without hose holder function is known, which serves for the connection of artificial kidneys. With the described rotating-change support holder, an artificial kidney can be rotated through 180° during use, without kinking hoses which are connected to the artificial kidney in this case.

A further filter holder without hose-holder function is known from DE 10 2009 024 448 A1.

Finally, a hold element for hose clamping assemblies in medical equipment is known from WO 2011/073766 A1.

### SUMMARY OF THE INVENTION

Therefore, an object of the invention is to provide a rugged filter holder with which hoses can be held in the area of a filter.

A further task of the invention is to provide a medical device with such a filter holder.

According to aspects of the present invention, this object is solved by a filter holder, in accordance with the independent Claim 1. Useful further developments of the filter holder result from the subordinate Claims 2-9. The task is further solved by a medical device for extra-corporal blood treatment in accordance with Claim 10.

The invention-related filter holder supporting a filter is used for extra-corporal blood treatment, where the filter holder indicates at least a first grip leg/limb and a second grip leg/limb, which are arranged with respect to each other so that a reception area is formed between the two grip legs for the support of a filter on the casing of a medical device. The filter can then be inserted into this reception area between the two grip legs/limbs, so that the filter is held in the filter holder against the force of gravity at least.

According to aspects of the present invention, at least one trough-shaped indentation/concavity is provided on at least one grip leg, whose cross-section enables the clamping-in of a hose. This at least one indentation/concavity thus forms a hose bracket/holder on the filter holder. As a result of this, the filter holder also fulfills the requirement of a hose bracket, as well as the function of a filter holder in the area of the filter, where no additional component parts are necessary. Rather, the hose bracket is integrated into the filter holder geometry and can be represented/formed cost-neutrally.

Furthermore, it involves a one-piece solution, which does not require any interfaces to other component parts. This also increases patient security, since the hose bracket is (integrally) attached captive on the filter holder and hoses can be sorted and oriented very near to the filter. The at least one hose bracket is integrated into the appearance of the grip legs of the filter holder in this case, and therefore does not disturb the overall appearance.

Preferably, the at least one trough-shaped indentation/concavity is located on an outer side of the grip leg/limb which is directed away from the receptacle area. The hose bracket in this case is formed outside on the filter holder. However, it could also be formed on an inner side of the filter holder, so that one or more hoses are also capable of being be inserted into an area between the two grip legs/limbs, if this is useful in case of certain applications and if sufficient room is provided there.

In one embodiment of the invention, two trough-shaped indentations/concavities are provided on at least one grip leg/limb, so that two hose brackets are formed. Also a higher number of indentations/concavities is possible. Furthermore, at least one trough-shaped indentation/concavity can extend perpendicular to the longitudinal alignment of a grip element. This then typically leads to a vertical orientation of a hose, if the grip legs/limbs extend horizontally. However, one or more hose brackets can also be aligned/orientated in other or even different directions. Thus hoses can also be directed in different directions if required. In addition, the indentations/concavities of the hose brackets can also indicate different cross-sections, so that hoses of different sizes can be clamped in.

The at least one grip leg/limb can be formed from a metal profile as it is often employed for filters in the area of extra-corporal blood treatment. In particular, it can involve an extruded section, for example of aluminum.

In order to enable the reception and holding of a filter in the reception area between the two grip legs/limbs, the geometry and the mechanics of the filter holder can be designed as desired. In a preferred embodiment of the invention, the first grip leg is supported resistant to rotation on the second grip leg, so that the two grip legs form a type of forceps with which a filter can be held. This can be implemented in particular through friction. However, filter and filter holder can be additionally or alternatively also formed with respect to each other, so that a form closure/form fit is used for the holding.

On the first grip leg/limb, which is supported so that it can be rotated, for example a spring force can be applied, which can be converted into a forceps-type movement of the first grip leg/limb in the direction of the second grip leg/limb. In order to generate this spring force and to correspondingly convert it, the second grip leg/limb can, for example, be assembled on an axis or shaft on which an axially-movably, plain-bearing bush is supported in turn. In this case, the plain-bearing bush is spring-loaded, where by a permanent pressure force acts on an area of the first grip leg/limb, which is formed such that the pressure force can be converted into a forceps-type movement of the first grip leg/limb in the direction of the second grip leg.

Further included in the invention is a medical device for extra-corporal blood treatment, which includes a filter holder on the casing of the device, wherein the filter holder is formed in accordance with the present invention. Such a medical device has the advantage in particular that no disturbing or sensitive hose brackets have to be provided on the casing for the area of the dialyzer, since the filter holder already fulfills the function of the tube/hose support.

### BRIEF DESCRIPTION OF THE DRAWINGS

The invention is best understood from the following detailed description when read in connection with the accompanying drawings. Included in the drawings are the following figures:

FIG. 1 A schematic view of an embodiment of the filter holder according to the invention,

FIG. 2 A schematic view of the filter holder, in accordance with FIG. 1, with inserted filter and hose.

### DETAILED DESCRIPTION OF THE PREFERRED EMBODIMENTS

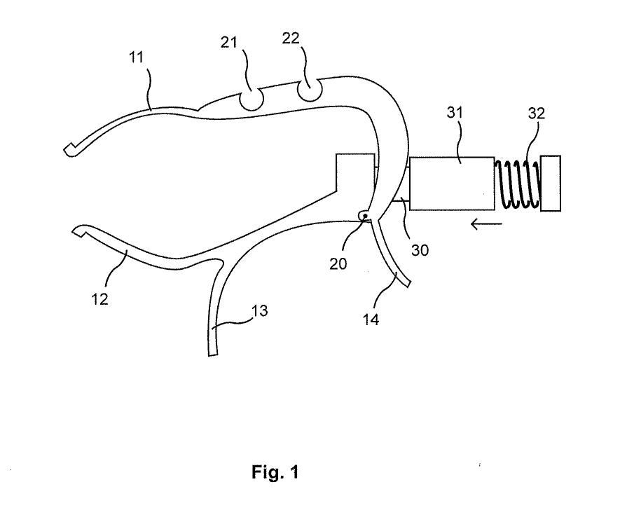

FIG. 1 indicates a design example of the filter holder 10 according to the invention without inserted filter. In the case of a filter to be held, it can involve for example the dialyzer of a medical device for extra-corporal blood treatment wherein the filter holder 10 is also designated as a dialyzer holder in this case.

In order to enable the reception of a filter in the filter holder 10, two grip legs/limbs 11 and 12 are provided, between which a reception area is formed for the incorporation of the filter. These grip legs/limbs can also be designated as dialyzer clamps if it involves a dialyzer in case of the filter. For the reception of a filter, the grip legs 11, 12 are implemented curved inwards (concave) in a grip area, in order to enable the easy grasping of a cylinder-shaped filter and to enable it to be held. The inner surfaces of the grip legs/limbs 11, 12 can furthermore be implemented fluted, or indicate a special surface with high friction coefficient.

A first grip leg 11 is supported, resistant to rotation, at a pivot 20 on the second grip leg 12. Preferably, this is implemented spring-loaded so that a spring force causes a forceps-type movement of the first grip leg 11 in the direction of the second grip leg 12, such that a filter can be wedged in between the two grip legs 11, 12. The spring force is applied for example by a plain-bearing bush 31, which is supported axially movable on an axis or shaft 30, which in turn is fixed-connected with the second grip leg 12. By a compression spring 32, a permanent force acts to the left on the plain bearing bush 31, as is shown by an arrow in FIG. 1. As a result of this spring force, a permanent pressure force acts on the outer contour of the first grip leg 11 which is formed arch-shaped at this point so that this pressure force can be converted into a forceps-type movement of the first grip leg 11 in the direction of the second grip leg 12.

Furthermore, a first spreading grip 14 is formed on the first grip leg 11 in the area of the pivot 20, while a second spreading grip 13 is formed on the second grip leg 12. The two spreading grips 13, 14 are formed and arranged in this case so that they can be grasped by an operator and simultaneously closed on each other. As a result of this movement, the filter holder 10 is opened as the first grip leg 11 is pivoted with respect to the second grip leg 12 outwards around the pivot point 20. As a result of this, the separation distance between the two grip legs 11, 12 is increased and a filter can be inserted into the reception area between the two grip legs 11, 12.

On the first grip leg 11 at least one trough-shaped indentation/concavity is inserted into the profile section of the grip leg 11, wherein, in the embodiment as shown in the figures, two such indentations 21 and 22 are provided. These indentations 21, 22 form hose brackets on the grip leg 11, into which hoses can be clamped. The hose brackets in this case are preferably located on the outer side of the grip leg 11, however, they can also be attached/provided for example on the inner side thereof. In addition, hose brackets can also be formed on the second grip leg 12.

Furthermore, the trough-shaped indentations/concavities 21, 22 preferably have a substantially round cross-section, however any arbitrary cross-sections can also be selected, as long as they are suited for a clamping support of hoses. For this purpose, edges should be provided on the boundary surface between a grip leg and the inner surface of the indentation, which reduce the opening of the hose bracket to such an extent that a hose is slightly squeezed when inserted. Thus the hose is wedged and held within the indentation/concavity.

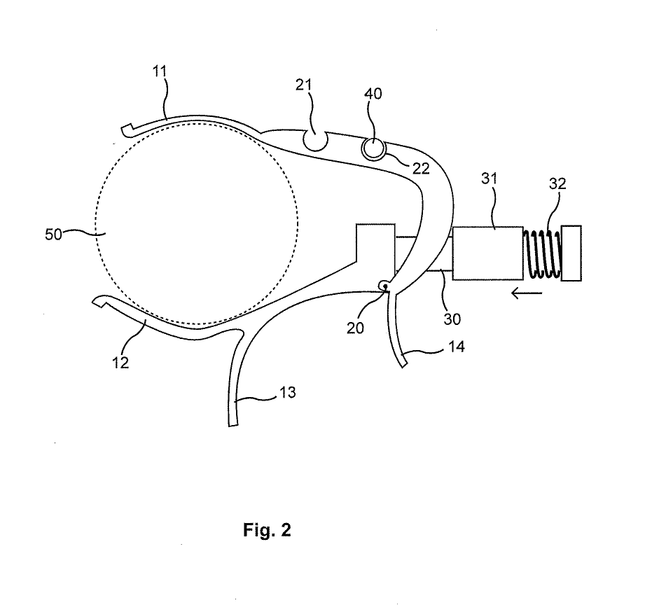

FIG. 2 indicates a schematic view of an embodiment of the invention-related filter holder 10 with an inserted filter 50, whose cross-section is shown by dashed lines. The grip legs 11, 12 of the filter holder 10 were spread apart for this purpose, so that the filter 50 could be inserted from the left into the reception area between the two grip legs 11, 12. For this procedure, the front ends of the two grip legs are implemented for example slightly convex/curved outwards, in order to facilitate a guiding of the filter with round cross-section into the reception area.

As a result of the pivot movement of the first grip leg 11, the plain-bearing bush 31 was displaced to the right on the axis 30, so that the pressure force by the spring 32 on the plain-bearing bush 31 has been increased. In this way the filter 50 can be wedged in between the two grip legs 11, 12. The filter 50 can be additionally be shaped so that it gets, for example, a surrounding concavity/rejection at which it is supported on the upper side of the grip legs/limbs 11, 12 and thus secured against sliding down.

Furthermore, it is indicated in FIG. 2 how a hose 40 is inserted into the indentation/concavity 22. In this case, the indentation/concavity 22 extends perpendicular to the longitudinal alignment of the grip leg 11, so that the hose 40 is aligned parallel to the longitudinal axis of the filter 50 on the filter holder 10.

1. Filter holder for the support of a filter on the casing of a medical device for extra-corporal blood treatment, wherein the filter holder comprises: a first grip leg and a second grip leg, which are arranged with respect to each other such that a reception area is formed between the two grip legs, and the filter is capable of being brought into this reception area between the two grip legs, so that the filter is held in the filter holder against the force of gravity wherein on at least one grip leg at least one trough-shaped indentation or concavity is provided, whose cross-section is adapted for wedging in of a hose.

2. Filter holder according to claim 1, wherein at least one trough-shaped indentation or concavity is located on an outer side of the grip leg, which outer side is directed away from the reception area.

3. Filter holders according to claim 1, wherein two trough-shaped indentations or concavities are provided on at least one grip leg.

4. Filter holder according to claim 1, wherein at least one grip leg is formed from a metal profile.

5. Filter holder according to claim 4, wherein the metal profile is an extruded section.

6. Filter holder according to claim 1, wherein at least one trough-shaped indentation or concavity extends perpendicular to the longitudinal alignment of a grip element.

7. Filter holder according to claim 1, wherein the first grip leg is supported resistant to rotation on the second grip leg.

8. Filter holder according to claim 7, wherein a spring force is applied on the first grip leg being pivotingly supported, which spring force can be converted into a forceps-type movement of the first grip leg in the direction to the second grip leg/limb.

9. Filter holder according to claim 8, wherein the second grip leg is assembled on an axle, on which a plain bearing bush is supported axially movable, wherein the plain bearing bush is spring-loaded, wherein a permanent pressure force acts on an area of the first grip leg, which is formed such that the pressure force can be converted into a forceps-type movement of the first grip leg in the direction to the second grip leg.

10. Medical device for extra-corporal blood treatment, including a filter holder on the casing of the device, wherein the filter holder is formed in accordance with claim 1.
